(19) United States
(12) Patent Application Publication
(10) Pub. No.: US 2026/0069658 A1
(43) Pub. Date: Mar. 12, 2026

(54) Engineered Ligand promotes TREM1-dependent anti-tumor immunity through cross-presentation

(71) Applicant: National Yang Ming Chiao Tung University, Taipei (TW)

(72) Inventors: Nien-Jung Chen, New Taipei City (TW); Chien-Jung Li, Tainan City (TW)

(21) Appl. No.: 19/240,780

(22) Filed: Jun. 17, 2025

Related U.S. Application Data
(60) Provisional application No. 63/660,772, filed on Jun. 17, 2024.

(51) Int. Cl. A61K 38/17; A61P 35/00

(52) U.S. Cl. CPC A61K 38/1738; A61P 35/00

(57) ABSTRACT

The present invention provides a recombinant multivalent TREM1 agonist, termed Tetra-CNX, designed to engage and activate TREM1 on myeloid cells. The ligand comprises multiple calnexin luminal domains assembled in a multimeric configuration, enabling high-avidity receptor binding and downstream SYK-dependent signaling. Upon activation, Tetra-CNX enhances lysosomal remodeling and promotes efficient antigen uptake, processing, and presentation via MHC class I and II pathways. This invention enables improved priming of CD4⁺ and CD8⁺ T cells and facilitates immune activation in contexts requiring enhanced antigen presentation, including but not limited to cancer, infectious diseases, and vaccine responses. Pharmaceutical compositions and methods of use are provided for modulating myeloid cell function and adaptive immunity through TREM1-targeted intervention.

### CROSS-REFERENCE TO RELATED APPLICATION

The present application claims benefit of priority to U.S. Provisional Patent Application No. 63/660,772 entitled “Soluble recombinant peptide for tumor immune monitoring and use thereof” and filed on Jun. 17, 2024, which is specifically incorporated by reference herein for all that it discloses or teaches.

### REFERENCE TO AN ELECTRONIC SEQUENCE LISTING

The application contains a Sequence Listing which has been submitted electronically in .XML format and is hereby incorporated by reference in its entirety. Said. XML copy, created on Jun. 16, 2025, is named “T2847-US-02_SLxml” and is 7,000 bytes in size. The sequence listing contained in this XML file is part of the specification and is hereby incorporated by reference herein in its entirety.

### FIELD OF THE INVENTION

The present invention relates to engineered recombinant ligands and their application in modulating immune responses within the tumor microenvironment. Specifically, the invention provides soluble peptide-based compositions derived from calnexin (Tetra-CNX) that act as agonists of TREM1 signaling in tumor-associated myeloid cells. These compositions are capable of enhancing both MHC class I- and MHC class II-mediated antigen presentation via a SYK-dependent pathway, leading to improved activation of CD8⁺ and CD4⁺ T cells. The invention further includes methods of using such ligands to reprogram tumor-associated macrophages (TAMs) to support antitumor immunity, either alone or in combination with checkpoint inhibitors and other immunotherapeutic agents.

### BACKGROUND OF THE INVENTION

### Tumor Antigen Presentation and Myeloid Plasticity

Effective antitumor immunity depends on the coordinated interaction between myeloid and lymphoid cells to recognize, process, and eliminate malignant cells. Professional antigen-presenting cells (APCs), such as dendritic cells and macrophages, are essential for capturing tumor-derived antigens—including neoantigens arising from somatic mutations—and presenting them via major histocompatibility complex (MHC) molecules to initiate adaptive T cell responses. In particular, the ability to cross-present exogenous antigens on MHC class I molecules is crucial for the priming of cytotoxic CD8⁺ T cells, and is increasingly attributed to subsets of tumor-infiltrating macrophages under specific activation conditions.

### Immunosuppressive Tumor Microenvironment and TAM Reprogramming

Despite this capacity, the tumor microenvironment (TME) frequently imposes an immunosuppressive state on myeloid cells, diminishing their antigen-presenting function. Tumor-associated macrophages (TAMs) are often characterized by tolerogenic, anti-inflammatory, or protumoral phenotypes. However, recent studies have revealed that TAMs possess remarkable phenotypic plasticity and can be reprogrammed to acquire immunostimulatory features, including enhanced antigen processing and T cell priming, in response to appropriate molecular cues.

### Reconsidering the Role of TREM1 in Tumor Immunity

Triggering Receptor Expressed on Myeloid Cells 1 (TREM1) is a cell-surface immunoreceptor traditionally associated with amplification of pro-inflammatory responses. Historically considered a negative prognostic factor in cancer, TREM1 has been proposed as a target for therapeutic inhibition. However, recent preclinical studies—including those using the TREM1 agonist antibody PY159—have demonstrated that activating TREM1 can promote beneficial immune responses within the TME. These observations suggest a previously underappreciated role for TREM1 signaling in supporting antitumor immunity, particularly through modulation of myeloid cell function.

### Need for Ligand-Based Agonists to Stimulate Myeloid Immunity

To date, few endogenous or engineered ligands capable of activating TREM1 in a context-specific and immunostimulatory manner have been identified. There remains a need for therapeutic strategies that harness TREM1 activation to enhance antigen presentation, especially within suppressive tumor niches. Such approaches may offer a complementary mechanism to checkpoint blockade and other immunotherapies.

### SUMMARY OF THE INVENTION

The present invention provides compositions and methods for enhancing tumor antigen presentation through activation of the TREM1 (Triggering Receptor Expressed on Myeloid Cells 1) pathway. In particular, the invention discloses a recombinant multimeric calnexin-derived ligand, herein referred to as Tetra-CNX, which selectively binds TREM1 and reprograms tumor-associated macrophages (TAMs) toward an antigen-presenting and immunostimulatory phenotype.

Tetra-CNX engages TREM1 and promotes its internalization, leading to SYK-mediated downstream signaling and trafficking into functional endolysosomal compartments. This process enhances both MHC-I and MHC-II-restricted antigen presentation by macrophages, increases lysosomal acidification and antigen digestion capacity, and upregulates key costimulatory molecules such as CD86.

In tumor-bearing mice, administration of Tetra-CNX increases the expression of MHC-I-peptide complexes on TAMs, elevates MHC-II and CD86 levels, and induces robust infiltration of CD8⁺ and CD4⁺ T cells into the tumor microenvironment. These effects are dependent on TREM1 and are abolished in Trem1-deficient models, underscoring the specificity of the ligand-receptor interaction.

Accordingly, the present invention provides:
a soluble TREM1 ligand composition for use in immunotherapy,
methods of enhancing intratumoral antigen presentation via TREM1 activation, and
uses of Tetra-CNX for priming or augmenting T cell responses in cancer.

These findings reveal a context-specific, immunostimulatory function for TREM1 and support the use of engineered TREM1 ligands as immunotherapeutic agents, either alone or in combination with checkpoint inhibitors or antigen-specific vaccines.

### BRIEF DESCRIPTION OF THE DRAWINGS

FIG. 1 is a structural model depicting the interaction between the calnexin ligand domain (Calnexin^{LD}) and the extracellular domain of human TREM1 (hTREM1^{ECD}), including predicted binding interfaces (a) and docking simulations using RxDOCK (b). Docking analysis results using RxDOCK, with score versus RMSD distribution, indicating the stability and quality of predicted docking conformations (c).

FIG. 2 shows a pull-down assay using recombinant TREM1-Fc protein to assess binding specificity of calnexin and its domain variants, demonstrating that the calnexin ligand domain (Cnx^{LD}) interacts selectively with TREM1 (a). Compare truncated domains of calnexin, revealing that the interaction is predominantly mediated through the N-terminal domain (Cnx^{N}) rather than the P-domain (Cnx^{P}). A schematic representation of calnexin truncations is included (b).

FIG. 3 illustrates surface plasmon resonance (SPR) sensorgrams showing the binding affinity of calnexin to murine TREM1 (a) and human TREM1 (b), with calculated dissociation constants (K_{D}) in the nanomolar range.

FIG. 4 depicts the schematic structure of a tetrameric TREM1 ligand complex (Tetra-CNX) assembled via StrepTagII-StrepTactin interactions (a), and native PAGE analysis showing distinct multimeric forms compared to the monomeric calnexin construct (b).

FIG. 5 presents TREM1 internalization assays performed in 293T cells (a) and THP-1 cells (b), showing ligand-induced reduction of surface TREM1 levels upon treatment with multimeric calnexin (Tetra-CNX) compared to monomeric ligand and control.

FIG. 6 shows time-lapse confocal microscopy images of cells treated with DY649-labeled Tetra-CNX and expressing a BiFC reporter for TREM1-DAP12 interaction, demonstrating endosomal co-localization over time (a). Quantification of BiFC puncta (b) and internalized ligand signal (c) is presented alongside.

FIG. 7 shows immunoblot analysis of phospho-SYK (Tyr525/526) in RAW264.7 macrophages treated with Mono-CNX or Tetra-CNX over time, with or without TREM1 overexpression, demonstrating ligand-induced activation of SYK signaling.

FIG. 8 shows immunoblot analysis of site-specific SYK phosphorylation at Tyr323, Tyr352, and Tyr525/526 in THP-1 cells treated with Tetra-CNX, with or without overexpression of human TREM1 and pre-treatment with the endocytosis inhibitor Dynasore.

FIG. 9 shows confocal microscopy images of cells co-stained for BiFC signal (TREM1-DAP12), Lamp1, and DAPI, indicating increased co-localization of TREM1 signaling complexes with lysosomes after 30 minutes of Tetra-CNX treatment (a). Quantitative analysis of Pearson's correlation coefficients is provided (b).

FIG. 10 shows functional assays measuring lysosomal activity following Tetra-CNX treatment in cells with or without TREM1 overexpression and SYK inhibition. The panels include quantification of oxidative burst (Oxyburst-BSA) (a), lysosomal pH (b), and Lysotracker fluorescence intensity (c).

FIG. 11 illustrates antigen uptake efficiency measured by OVA-AF647 fluorescence in cells with or without human TREM1 overexpression and SYK inhibition, following Tetra-CNX treatment.

FIG. 12 shows antigen degradation analysis using DQ-OVA in cells treated with Tetra-CNX, with or without human TREM1 overexpression and SYK inhibition (a). Histograms and quantification of the antigen digestion index demonstrate TREM1- and SYK-dependent enhancement of antigen processing (b).

FIG. 13 shows antigen degradation analysis using DQ-OVA in wild-type and Trem1 knockout cells treated with Tetra-CNX or control (a). Histogram overlays and quantification of the antigen digestion index demonstrate that the effect of Tetra-CNX on antigen processing is dependent on the presence of TREM1 (b).

FIG. 14 displays single-cell transcriptomic analysis of tumor-infiltrating immune cells in control (ST) and Tetra-CNX-treated mice. Odds ratio analyses reveal treatment-associated shifts in the abundance of CD8⁺ T cells, CD4⁺ T cells.

FIG. 15 shows t-SNE plots of tumor-infiltrating CD4⁺ T cells classified into pre-activated, early activated, and effector subtypes, illustrating a shift toward more activated states following Tetra-CNX treatment.

FIG. 16 shows flow cytometry analysis of CD4⁺ T cell proliferation following co-culture with GM-BMMs pre-treated with control or Tetra-CNX (a). Proliferative enhancement is observed only in wild-type macrophages and not in Trem1 knockout cells, indicating a TREM1-dependent effect (b).

FIG. 17 shows CD4⁺ T cell proliferation following co-culture with Tetra-CNX-treated GM-BMMs in the presence or absence of a SYK inhibitor (a). Flow cytometry analysis and quantification confirm that Tetra-CNX-mediated enhancement of CD4⁺ T cell proliferation is abrogated by SYK inhibition (b).

FIG. 18 shows t-SNE plots of tumor-infiltrating CD8⁺ T cells categorized into stem-like, early activated, and effector/exhausted phenotypes. Tetra-CNX treatment increases the proportion of stem-like and early activated subsets, suggesting enhanced priming and preserved functional potential of CD8⁺ T cells.

FIG. 19 is a composite graph illustrating the effect of Tetra-CNX treatment on the composition of CD8⁺ T cell subsets in a tumor model. The left panel shows the distribution of stem-like, early activated, and effector/exhausted CD8⁺ T cells as a percentage of total CD8⁺ T cells under ST or Tetra-CNX treatment (a). The right panel presents the log₂ odds ratios of each subset between the two treatment conditions, indicating a significant enrichment of stem-like CD8⁺ T cells following Tetra-CNX administration (b).

FIG. 20 is a volcano plot depicting differential gene expression profiles of CD8⁺ T cells under Tetra-CNX versus ST treatment. Each dot represents an individual gene, with the x-axis indicating the log₂ fold change and the y-axis showing the negative logarithm of the P-value. Genes significantly upregulated or downregulated by Tetra-CNX treatment are labeled, highlighting key genes associated with T cell activation or exhaustion.

FIG. 21 shows CD8⁺ T cell proliferation following co-culture with Tetra-CNX-treated GM-BMMs from wild-type or Trem1⁻/⁻ mice (a). Only wild-type macrophages enhance CD8⁺ T cell proliferation, confirming that TREM1 expression in macrophages is essential for ligand-mediated CD8⁺ T cell priming (b).

FIG. 22 shows CD8⁺ T cell proliferation following co-culture with Tetra-CNX-treated GM-BMMs in the presence or absence of a SYK inhibitor (a). SYK inhibition abrogates Tetra-CNX-induced CD8⁺ T cell proliferation, confirming that this effect is dependent on SYK signaling downstream of TREM1 (b).

FIG. 23 shows tumor volume (a), tumor weight (b), and representative tumor images (c) in wild-type and Trem1⁻/⁻ mice bearing B16F10-SIINFEKL tumors. Mice were treated with ST, Mono-CNX, or Tetra-CNX. Tetra-CNX significantly suppressed tumor growth and reduced tumor weight in a TREM1-dependent manner, confirming its in vivo immunostimulatory and antitumor efficacy.

FIG. 24 shows flow cytometric quantification of tumor-associated macrophages (TAMs) positive for SIINFEKL-MHC-I complexes (% (a) and gMFI (b)) in WT and Trem1⁻/⁻ mice treated with ST, Mono-CNX, or Tetra-CNX. Tetra-CNX significantly increases SIINFEKL presentation on MHC-I in WT but not Trem1-deficient TAMs, confirming TREM1-dependent enhancement of tumor antigen presentation in vivo.

FIG. 25 shows gMFI of MHC-II (a) and CD86 (b) expression in tumor-associated macrophages (TAMs) from WT and Trem1⁻/⁻ mice treated with ST, Mono-CNX, or Tetra-CNX. Tetra-CNX treatment significantly upregulates MHC-II and CD86 expression in WT but not Trem1⁻/⁻ TAMs, indicating reprogramming of macrophages toward an immunostimulatory phenotype via the TREM1 pathway.

FIG. 26 shows the frequency of tumor-infiltrating CD8⁺ (a) and CD4⁺ T (b) cells in wild-type and Trem1-deficient mice treated with ST, Mono-CNX, or Tetra-CNX. Tetra-CNX significantly increased CD8⁺ and CD4⁺ T cell infiltration in wild-type mice, but this effect was lost in Trem1⁻/⁻ mice, indicating TREM1-dependent remodeling of the intratumoral immune landscape.

### DETAILED DESCRIPTION OF THE INVENTION

The following description is merely exemplary in nature and is in no way intended to limit the present teachings, application, of uses.

The present invention provides a novel and non-obvious approach to enhancing antitumor immunity by leveraging a recombinant, multivalent TREM1 ligand, herein referred to as Tetra-CNX. This engineered ligand effectively reprograms tumor-associated macrophages (TAMs) to promote antigen presentation and subsequent T cell activation within the immunosuppressive tumor microenvironment. Tetra-CNX exerts its effects through high-affinity receptor engagement, internalization via endocytosis, activation of SYK-dependent signaling pathways, and upregulation of lysosomal antigen-processing machinery. This cascade ultimately results in augmented presentation of tumor antigens on MHC class I and II molecules, thereby boosting both CD8⁺ and CD4⁺ T cell responses. The invention constitutes a significant advancement over existing immunomodulatory strategies by directly targeting and activating endogenous TREM1 in myeloid populations.

A key technical contribution of this invention is the identification of calnexin as a previously unrecognized ligand candidate for TREM1. This discovery enabled the rational design of Tetra-CNX as a functional, multimeric agonist. Using domain-specific biochemical interaction studies and molecular modeling, the present inventors elucidated a specific interaction interface between the luminal domain of calnexin and the TREM1 ectodomain. This interaction underpins the molecular mechanism through which the invention activates TREM1 signaling in vitro and in vivo, providing a foundation for translational applications in cancer immunotherapy.

The luminal domain of calnexin refers to the N-terminal extracellular/lumen-facing region of the calnexin protein that resides within the lumen of the endoplasmic reticulum (ER). This domain includes the carbohydrate-binding site responsible for recognizing monoglucosylated N-linked glycans on nascent glycoproteins and is critical for its chaperone function.

“Treatment” and “treating” refer to administration or application of a therapeutic agent to a subject or performance of a procedure or modality on a subject for the purpose of obtaining a therapeutic benefit of a disease or health-related condition.

“Subject” and “patient” refer to either a human or non-human, such as primates, mammals, and vertebrates. In particular embodiments, the subject is a human.

The term “therapeutically effective” as used throughout this application refers to anything that promotes or enhances the well-being of the subject with respect to the medical treatment of this condition.

Tumors for which the present treatment methods are useful include any malignant cell type, such as those found in a solid tumor, a hematological tumor, metastatic cancer, or non-metastatic cancer. Exemplary solid tumors can include, but are not limited to, a tumor of an organ selected from the group consisting of pancreas, colon, cecum, esophagus, gastrointestine, gum, liver, skin, stomach, testis, tongue, uterus, stomach, brain, head, neck, ovary, kidney, larynx, sarcoma, bone, lung, bladder, melanoma, prostate, and breast. Exemplary hematological tumors include tumors of the bone marrow, T or B cell malignancies, leukemias, lymphomas, such as, for example, diffuse large B cell lymphoma, blastomas, myelomas, and the like. Further examples of cancers that may be treated using the methods provided herein include, but are not limited to, carcinoma, lymphoma, blastoma, sarcoma, leukemia, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer and gastrointestinal stromal cancer), pancreatic cancer (including pancreatic ductal adenocarcinoma), glioblastoma, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, melanoma, superficial spreading melanoma, lentigo malignant melanoma, acral lentiginous melanomas, nodular melanomas, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; diffuse large B-cell lymphoma; mantle cell lymphoma; AIDS-related lymphoma; and Waldenstrom's macroglobulinemia), chronic lymphocytic leukemia (CLL), acute lymphoblastic leukemia (ALL), Hairy cell leukemia, multiple myeloma, acute myeloid leukemia (AML) and chronic myeloblastic leukemia.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

### EXAMPLES

### Example 1. Identification of TREM1-Calnexin Interaction

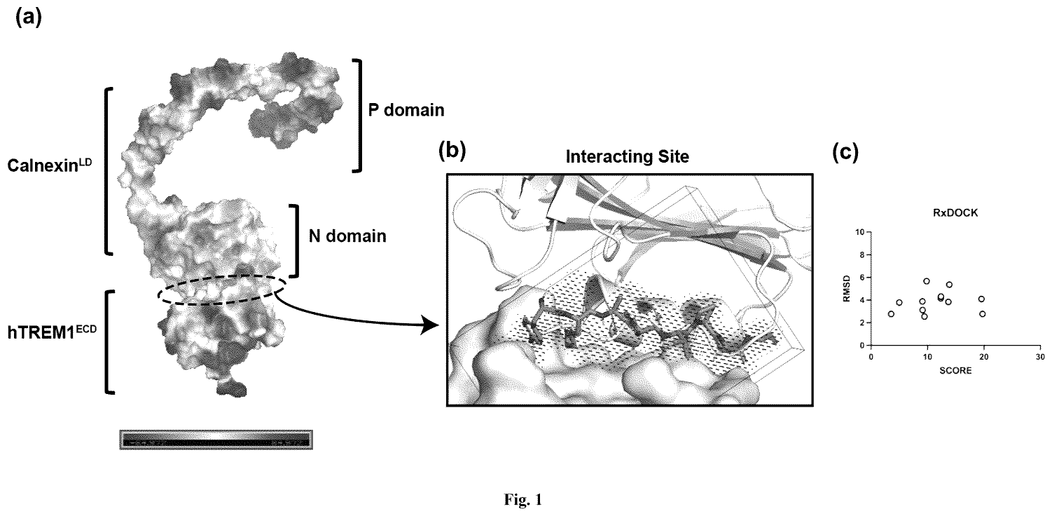

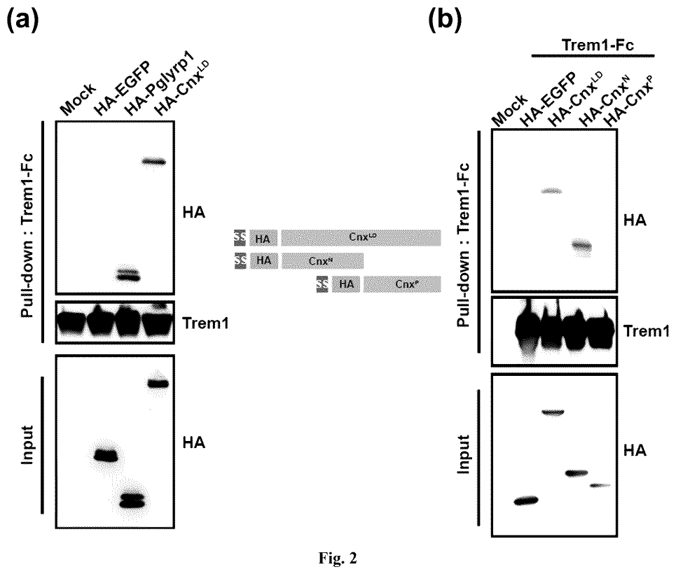

The inventors employed a BioID proximity labeling assay to identify proteins capable of interacting with the extracellular domain of TREM1. Expression of TREM1-BirA* in 293T cells followed by biotin supplementation and streptavidin pull-down yielded calnexin as a top-ranked interactor (Table 1). Supporting this discovery, in silico structural modeling indicated a plausible electrostatic interface between calnexin and TREM1 (FIG. 1). Co-immunoprecipitation studies using domain-specific calnexin truncations confirmed binding was mediated through the luminal domain, thus validating calnexin as a bona fide ligand candidate for TREM1 (FIG. 2).

Referred to Table 1, a comparative proteomic analysis showing proteins enriched by BirA*-TREM1 proximity labeling, identifying calnexin as a putative TREM1-interacting protein with selective enrichment relative to BirA*-TM control.

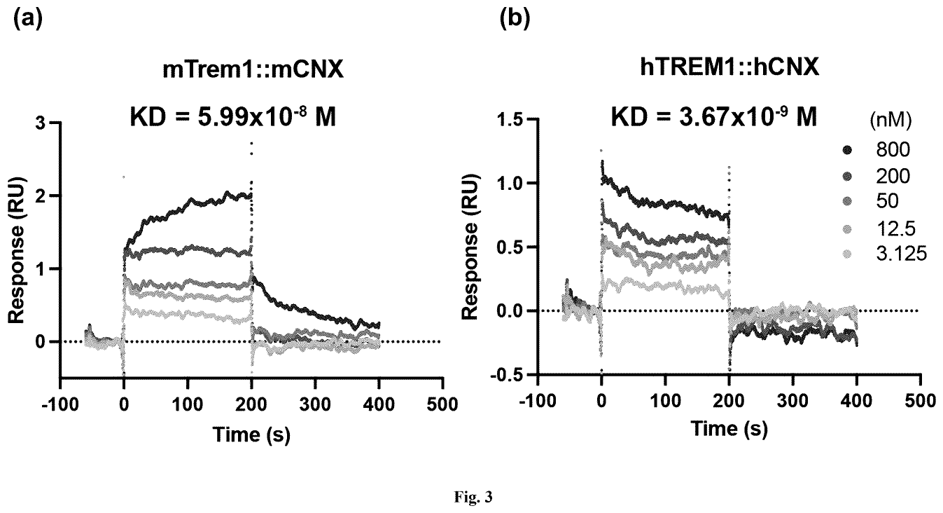

Surface plasmon resonance (SPR) analysis was performed to quantitatively assess the binding affinity between calnexin and TREM1 across species. Recombinant murine Trem1 ectodomain (mTrem1^{ECD}) and mouse calnexin luminal domain (mCNX^{LD}) were analyzed in a dose-dependent binding curve, demonstrating a dissociation constant (K_{D}) of 5.99×10⁻⁸ M (FIG. 3a). Similarly, recombinant human TREM1 and human calnexin (hCNX^{LD}) exhibited high-affinity interaction with a K_{D} of 3.67×10⁻⁹ M (FIG. 3b). These data confirm that calnexin functions as a conserved ligand for TREM1 in both mouse and human systems. Notably, the interaction exhibited high avidity and stable association phases, indicating potential for sustained receptor engagement in vivo.

TABLE 1

| | | | BirA*- |
|---|---|---|---|
| | | BirA*- | Trem1(21- |
| | | TM | 202)-TM |
| | | (T-SPC = | (T-SPC = |
| Protein | (kDa) | 7220) | 7136) |
| Calnexin | 67.5 | 0 | 2.0117713 |

### Example 2. Generation and Validation of Tetra-CNX

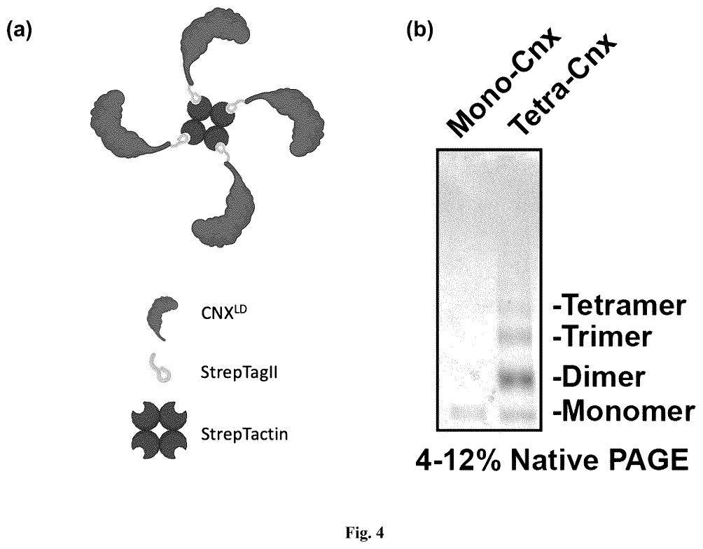

To construct a functional TREM1 agonist, the inventors developed Tetra-CNX, a tetrameric ligand composed of four calnexin luminal domains fused to StrepTagII and assembled via StrepTactin (FIG. 4a). This engineered construct was shown by native PAGE to form a defined multimeric complex (FIG. 4b).

### Example 3. TREM1 Internalization and Signal Transduction

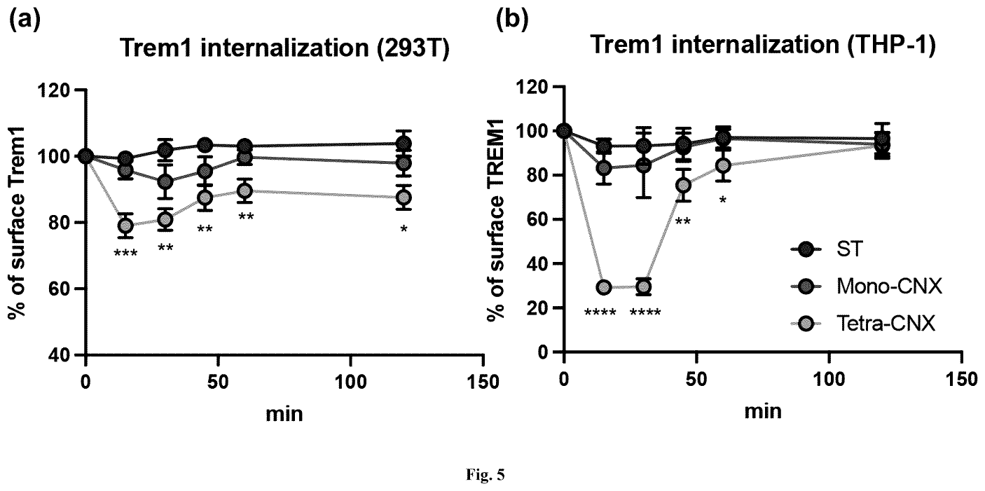

To investigate whether ligand-induced TREM1 internalization is ligand valency-dependent, the inventors assessed the surface expression of TREM1 following stimulation with either monomeric calnexin (Mono-CNX) or tetrameric calnexin (Tetra-CNX). Flow cytometry quantification of surface TREM1 levels revealed that Tetra-CNX treatment led to a significant and sustained reduction in cell surface TREM1 over time in both 293T cells (FIG. 5a) and THP-1 monocytic cells (FIG. 5b). In contrast, Mono-CNX induced only a moderate and transient reduction. These data demonstrate that multivalent TREM1 engagement by Tetra-CNX promotes robust receptor internalization, consistent with activation of downstream signaling cascades. The results further support that ligand multimerization is a critical determinant of TREM1 endocytosis dynamics and functional signal transduction.

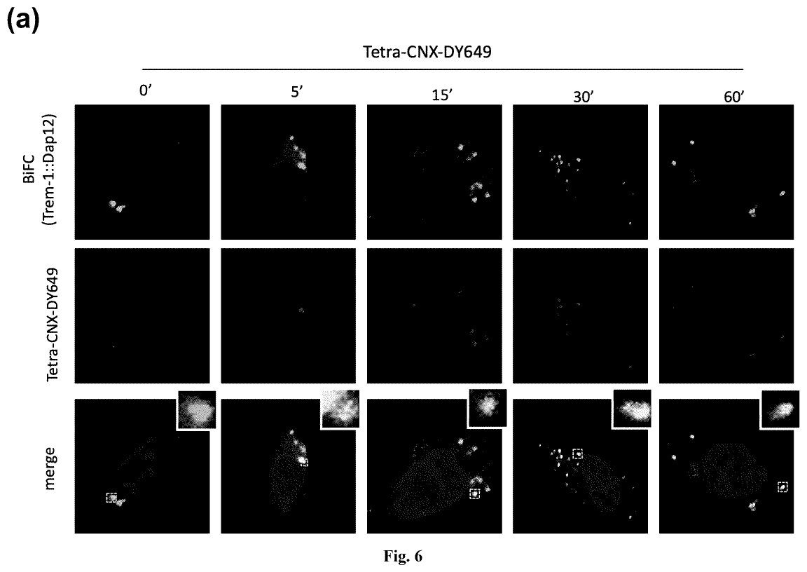

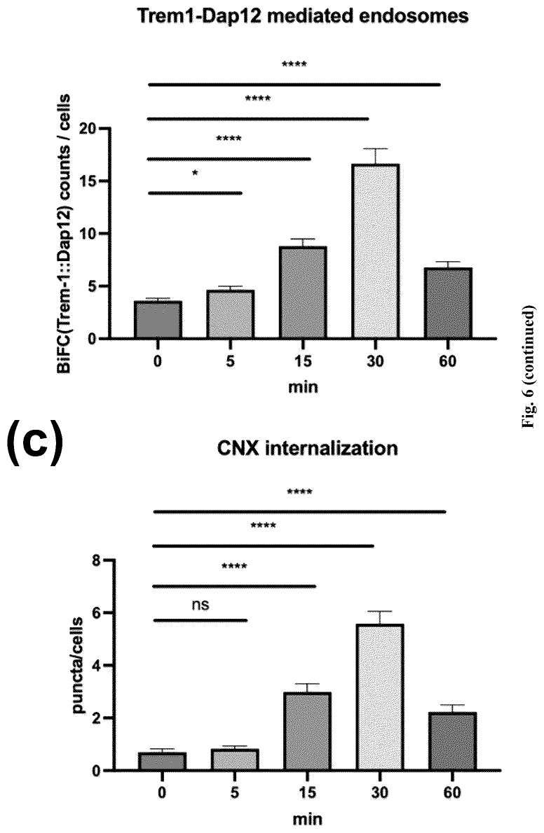

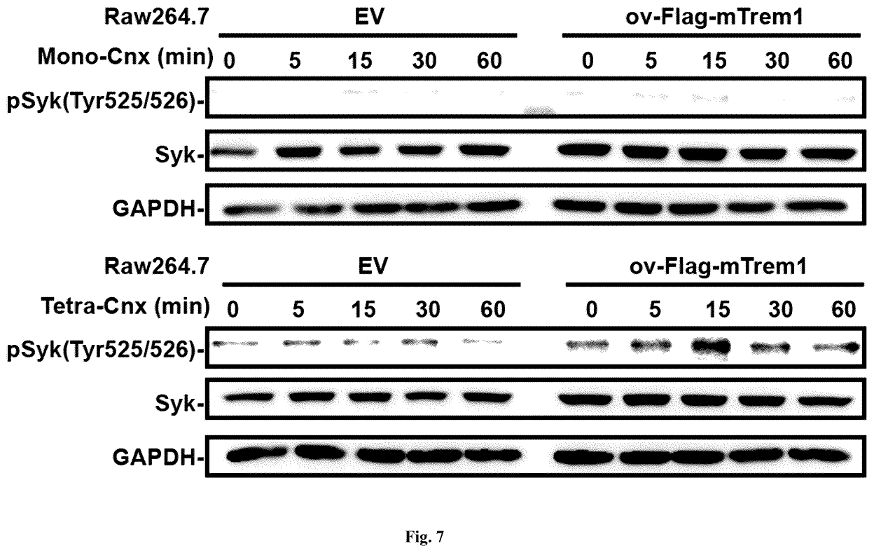

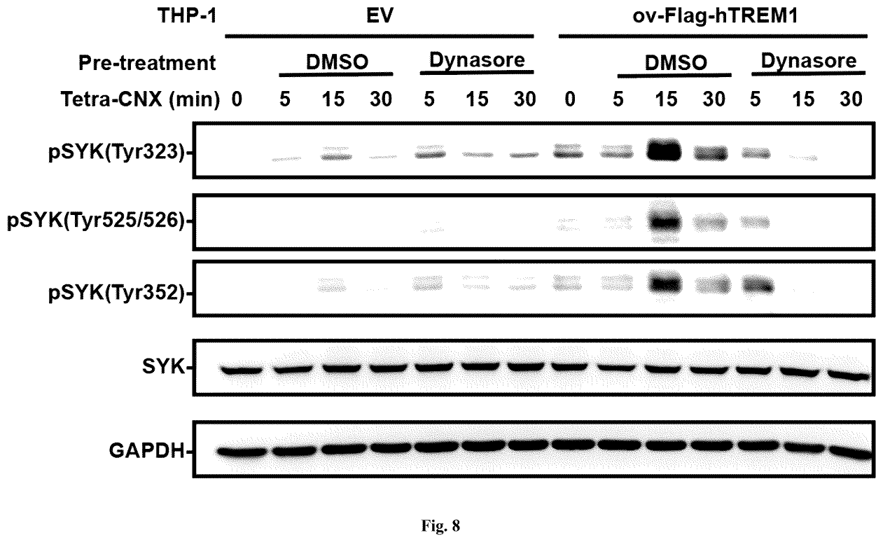

The inventors next evaluated the internalization and downstream signaling of TREM1 upon Tetra-CNX binding. BiFC analysis in CHO-K1 cells co-transfected with TREM1::YFP-N and DAP12::YFP-C demonstrated that Tetra-CNX treatment resulted in intracellular puncta, suggesting receptor clustering or internalization events (FIG. 6). Immunoblotting in RAW264.7 cells (FIG. 7) and THP-1 (FIG. 8) revealed rapid phosphorylation of SYK at Y525/526, which was reduced by dynamin inhibition. These findings support that Tetra-CNX activates canonical TREM1-DAP12-SYK signaling via a receptor-mediated internalization process.

### Example 4. Lysosomal Remodeling and Antigen Processing

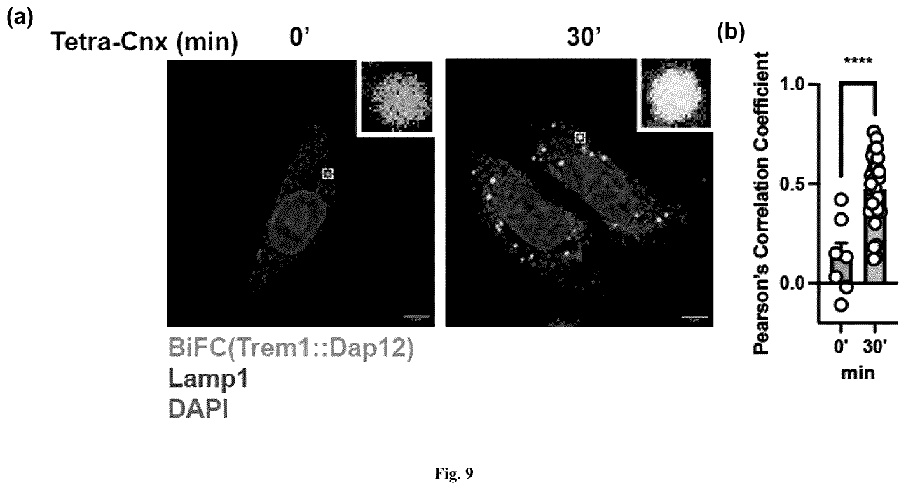

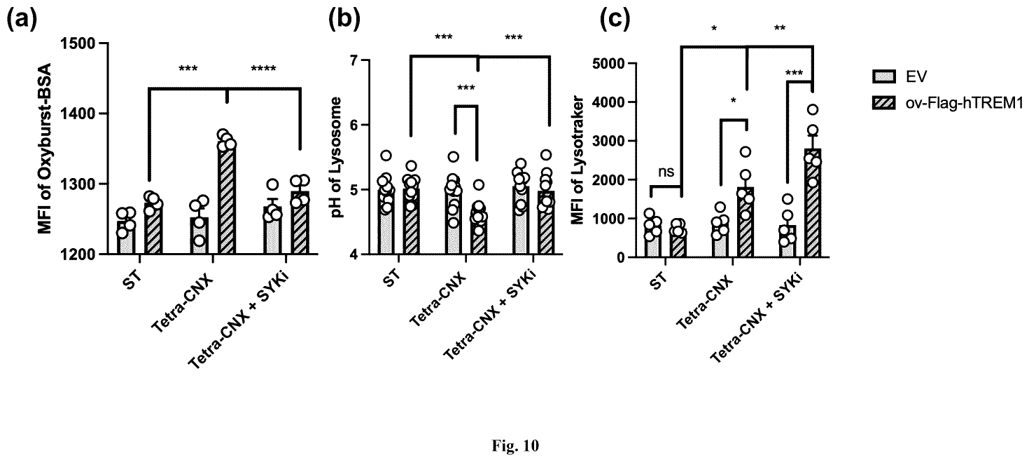

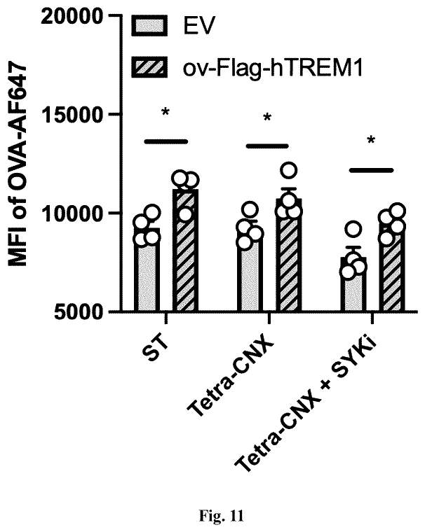

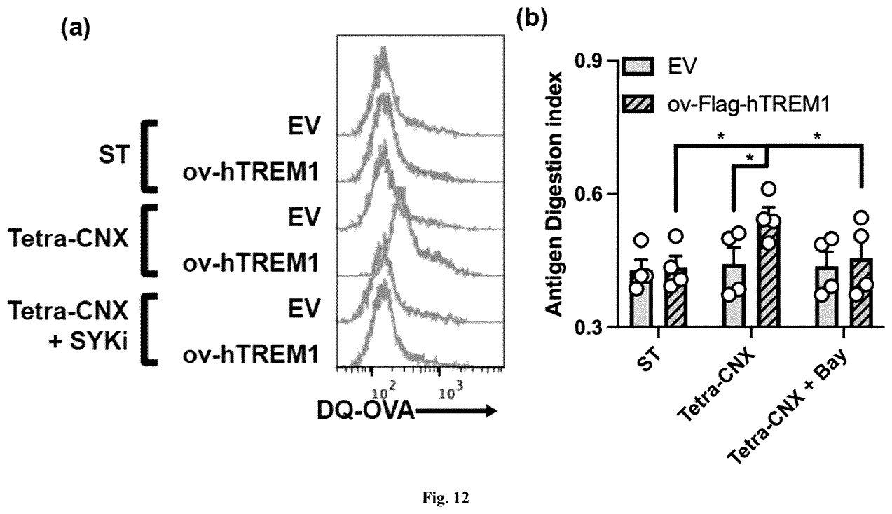

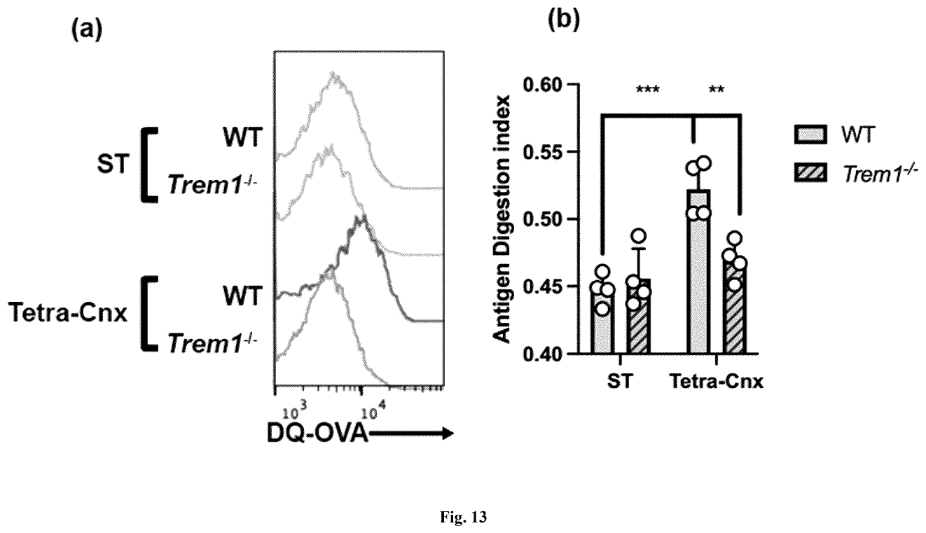

Upon ligand engagement, TREM1-DAP12 complexes contribute to the functional reprogramming of lysosomal activity (FIG. 9). The inventors utilized Oxyburst, LysoSensor, and LysoTracker probes to show that Tetra-CNX induces lysosomal oxidative stress, acidification, and volumetric expansion in TREM1-expressing macrophages (FIG. 10). Enhanced uptake (FIG. 11) and degradation (FIG. 12, 13) of model antigens (OVA-AF647 and DQ-OVA) were observed, and these effects were SYK-dependent. This example confirms that Tetra-CNX not only initiates signaling but also functionally reprograms endolysosomal processing to favor antigen presentation.

### Example 5. T Cell Activation and Antigen Presentation

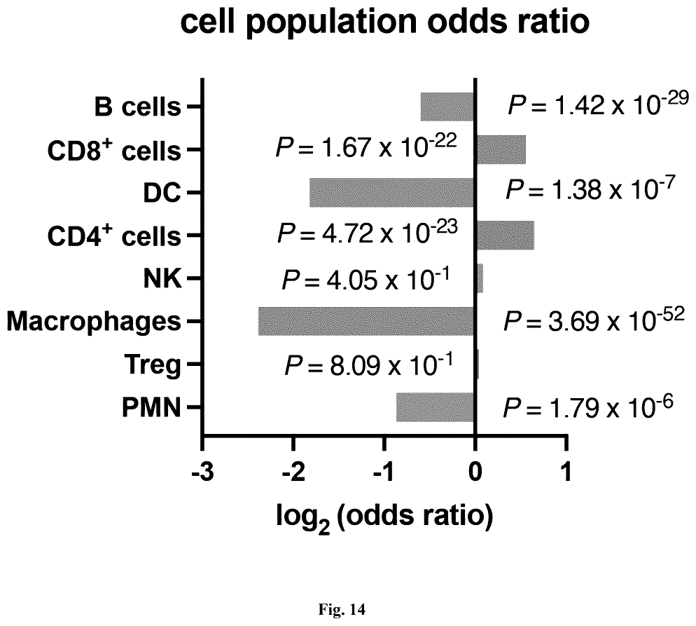

The present invention further demonstrates that administration of Tetra-CNX enhances antigen-specific T cell responses through reprogramming of macrophages. As shown by single-cell RNA sequencing analysis of tumor-infiltrating immune populations, Tetra-CNX treatment significantly increased the abundance of CD4⁺ and CD8⁺ T cells in vivo, indicating an altered immune composition favorable to antitumor immunity (FIG. 14)

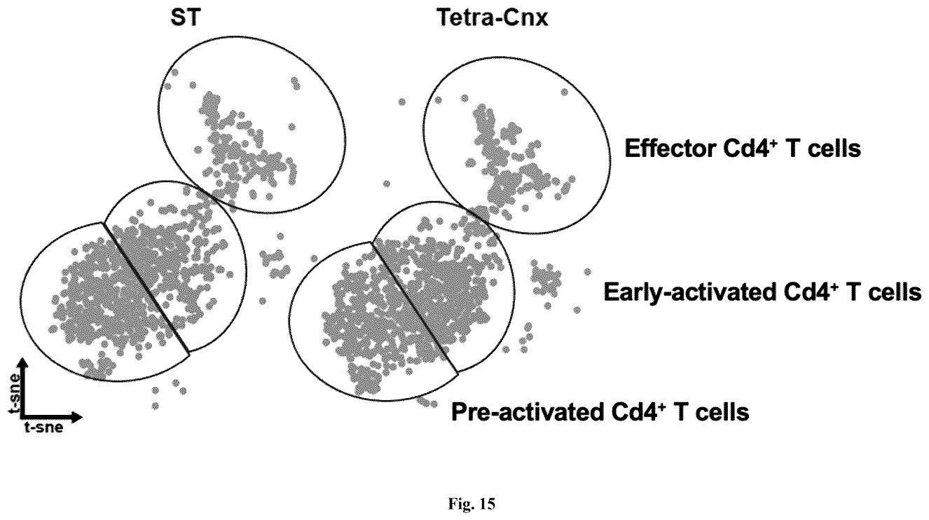

Single-cell transcriptomic projection and trajectory analysis further revealed that Tetra-CNX treatment increased the abundance of intratumoral CD4⁺ T cells and promoted their activation toward effector states (FIG. 15). Compared to the control group, Tetra-CNX not only expanded the pool of CD4⁺ T cells, but also facilitated their progression from pre-activated to early-activated and ultimately effector phenotypes. This transcriptional shift is indicative of an enhanced antigen presentation milieu within the tumor, supporting a functional reprogramming of helper T cells that favors antitumor immunity.

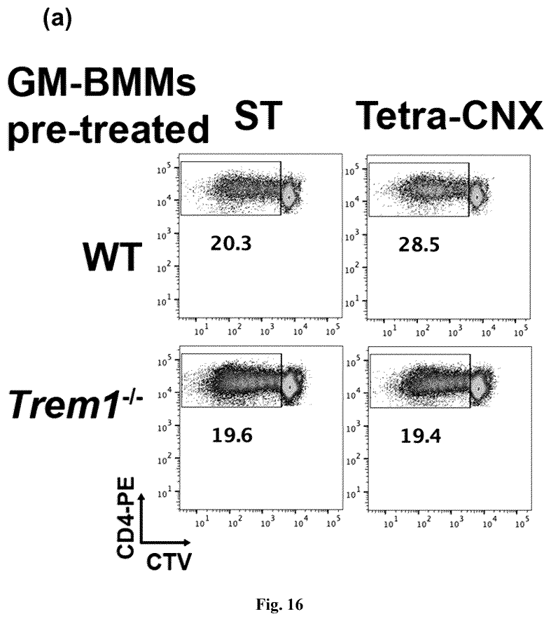

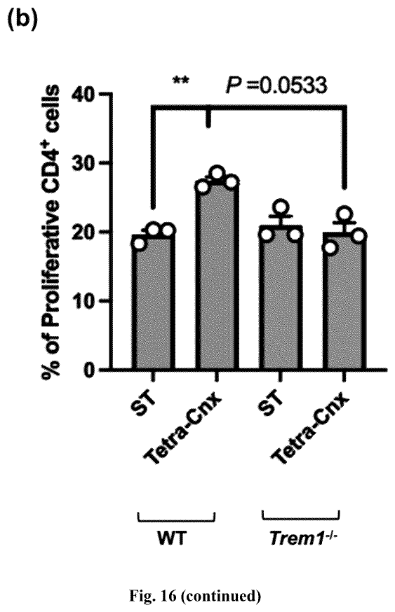

In a co-culture system, GM-CSF-derived bone marrow macrophages (GM-BMMs) pretreated with Tetra-CNX significantly enhanced the proliferation of naïve CD4⁺ T cells, as quantified by dilution of CellTrace Violet (CTV) dye (FIG. 16). Statistical significance was determined using unpaired two-tailed Student's t-test. *P<0.05 was considered statistically significant. This stimulatory effect was observed exclusively in wild-type macrophages, while TREM1-deficient macrophages failed to promote CD4⁺ T cell expansion regardless of Tetra-CNX treatment. These results confirm the functional requirement of TREM1 in Tetra-CNX-mediated enhancement of antigen presentation and helper T cell priming.

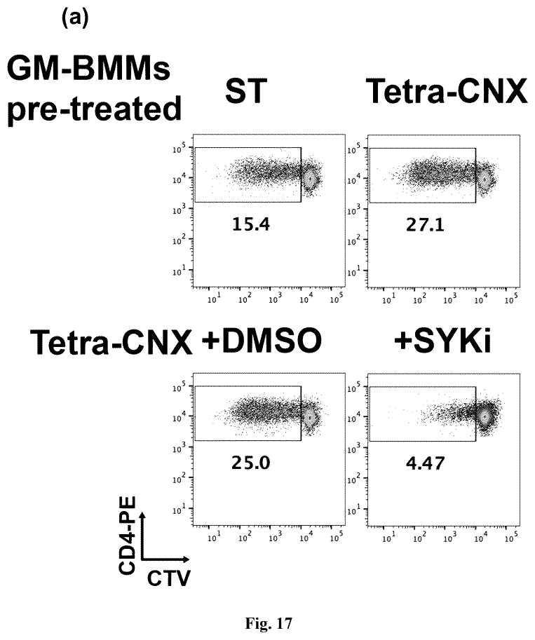

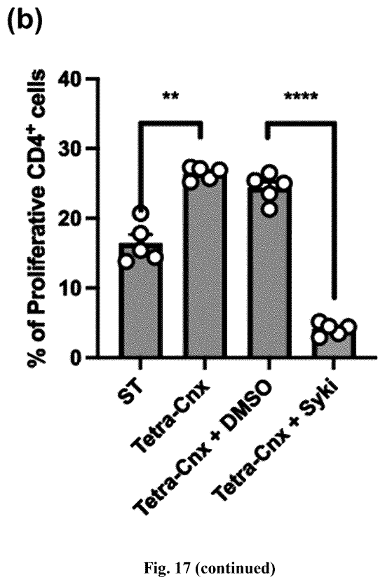

The requirement of SYK signaling in Tetra-CNX-induced CD4⁺ T cell priming was further examined using a selective SYK inhibitor (FIG. 17). Statistical significance was determined using unpaired two-tailed Student's t-test. **P<0.01 and ***P<0.001 were considered statistically significant. Inhibition of SYK abrogated the ability of Tetra-CNX-pretreated GM-BMMs to stimulate CD4⁺ T cell proliferation, reducing proliferative responses to baseline levels. These findings corroborate the essential role of TREM1-SYK axis in mediating the enhanced antigen presentation and helper T cell activation induced by Tetra-CNX.

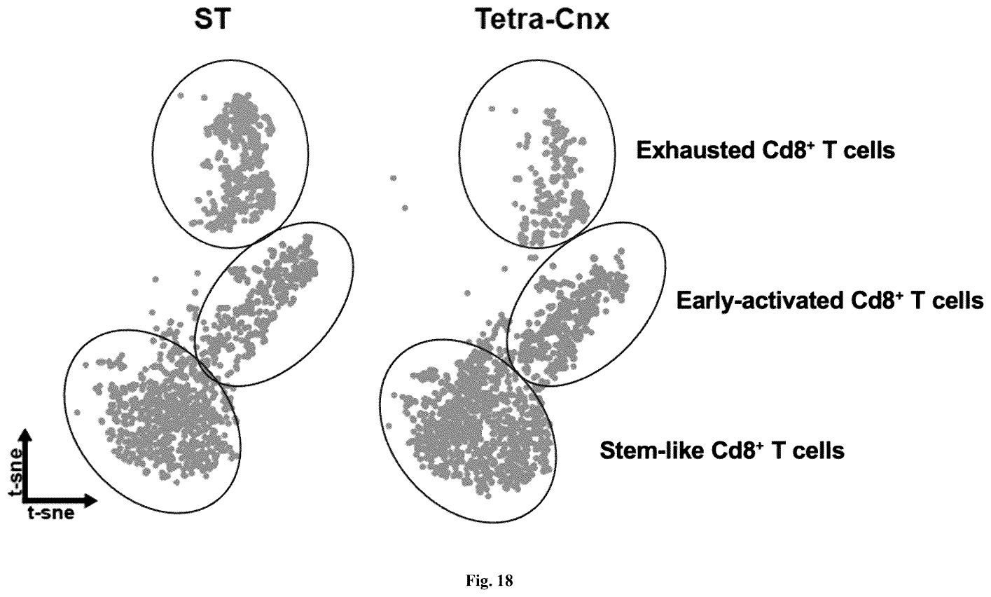

Single-cell transcriptomic analysis further revealed that Tetra-CNX treatment modulates the composition of CD8⁺ T cell subsets in vivo (FIG. 18). Specifically, Tetra-CNX promoted the expansion of stem-like and early-activated CD8⁺ T cells, while concurrently reducing the proportion of terminally exhausted CD8⁺ T cells. This shift in T cell state distribution suggests that Tetra-CNX-treated antigen-presenting cells not only increase CD8 T cell priming but also preserve their effector potential, contributing to more durable and functional antitumor immunity.

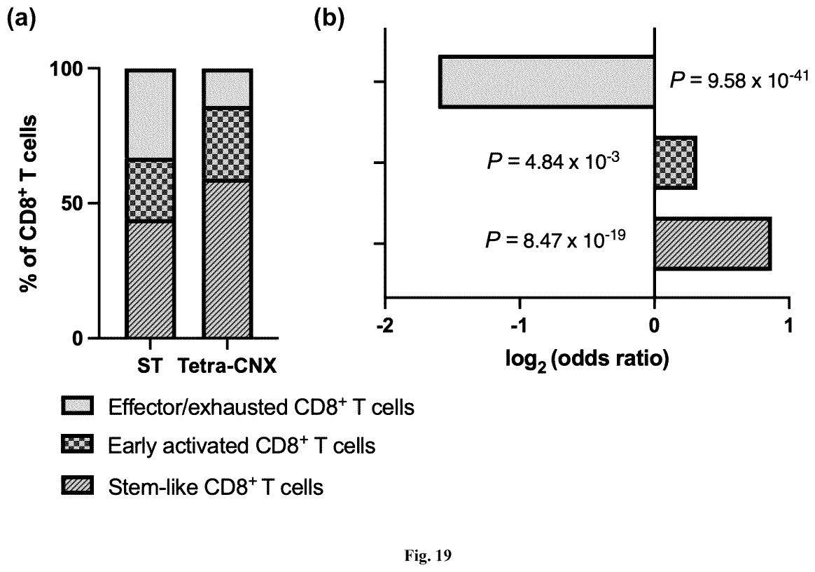

Single-cell transcriptomic analysis demonstrated that Tetra-CNX treatment significantly reshapes the intratumoral CD8⁺ T cell landscape (FIG. 19). Comparative t-SNE mapping and population deconvolution revealed an increased proportion of stem-like and early-activated CD8⁺ T cells, accompanied by a concomitant reduction in terminally exhausted CD8⁺ T cells in the Tetra-CNX-treated group. Quantitative odds ratio analysis confirmed the statistical enrichment of stem-like and early-activated CD8⁺ T cell subsets, with depletion of the exhausted subset. These results indicate that Tetra-CNX-induced antigen presentation not only enhances CD8⁺ T cell activation but also preserves their effector differentiation potential, offering a mechanistically distinct and immunologically advantageous profile compared to conventional stimulation. This shift in CD8⁺ T cell state composition underlines the therapeutic potential of Tetra-CNX in promoting durable and functional antitumor responses through modulation of antigen-specific cytotoxic immunity.

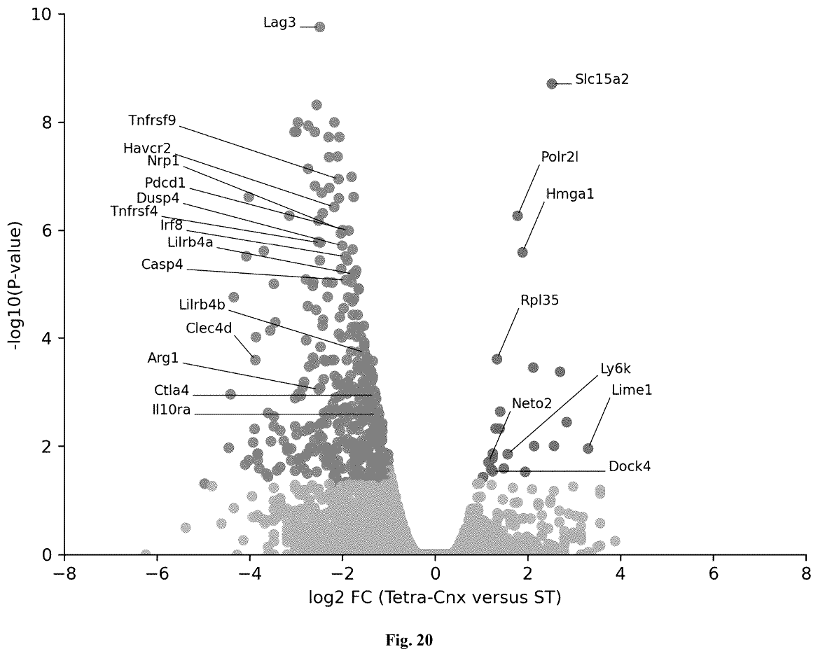

In some embodiments, transcriptomic profiling of tumor-infiltrating immune cells further supports the immunomodulatory effect of the CNX-based therapeutic. A volcano plot represents differentially expressed genes between Tetra-CNX-treated and ST-treated groups (FIG. 20). Each point corresponds to a gene, plotted by its log₂ fold change and statistical significance. Genes associated with T cell exhaustion (e.g., Lag3, Pdcd1, Haver2) are significantly downregulated, while genes linked to immune activation and cytotoxicity are upregulated. These results demonstrate that the disclosed CNX constructs not only reshape T cell states but also reprogram gene expression signatures relevant to antitumor immunity.

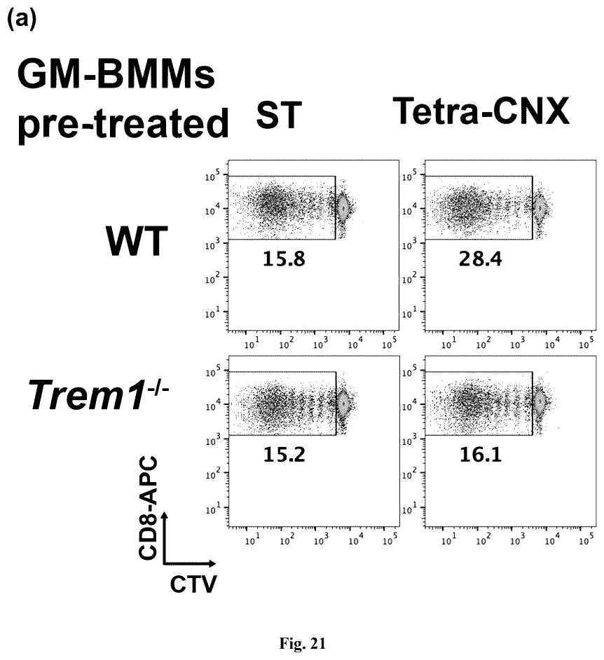

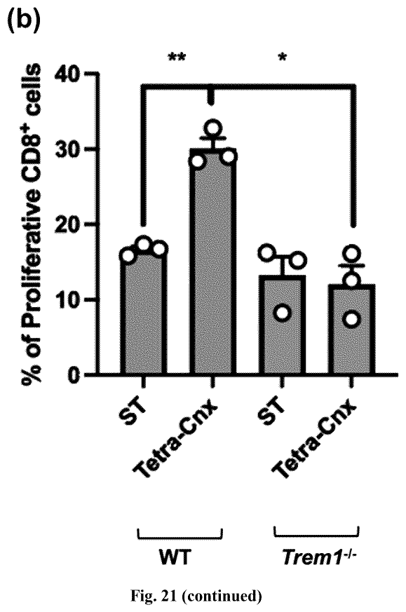

Co-culture experiments were conducted to assess the capacity of GM-CSF-derived bone marrow-derived macrophages (GM-BMMs) to promote CD8⁺ T cell proliferation following pre-treatment with either ST control or Tetra-CNX (FIG. 21). Data are presented as mean±SEM from three biological replicates. Statistical significance was determined using unpaired two-tailed Student's t-test. *P<0.05 and **P<0.01 were considered statistically significant. Wild-type macrophages pre-treated with Tetra-CNX significantly enhanced CD8⁺ T cell proliferation compared to the ST group, as evidenced by increased CTV dilution and quantified percentages. This T cell-stimulatory effect was abolished in Trem1⁻/⁻ macrophages, indicating a requirement for endogenous TREM1 expression in mediating the observed enhancement. These findings further validate the functional capacity of Tetra-CNX to license antigen-presenting myeloid cells in a TREM1-dependent manner, resulting in heightened CD8 T cell activation.

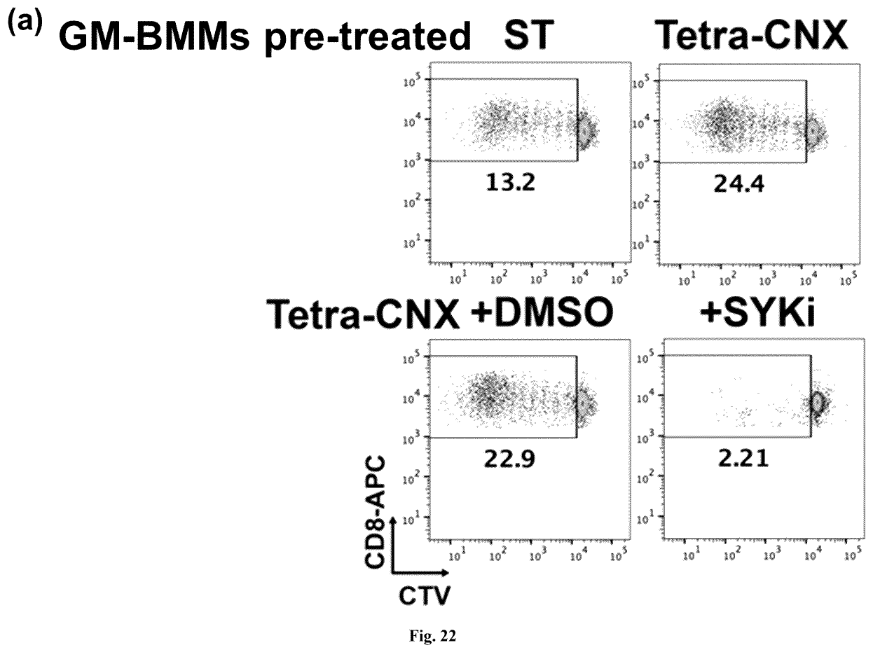

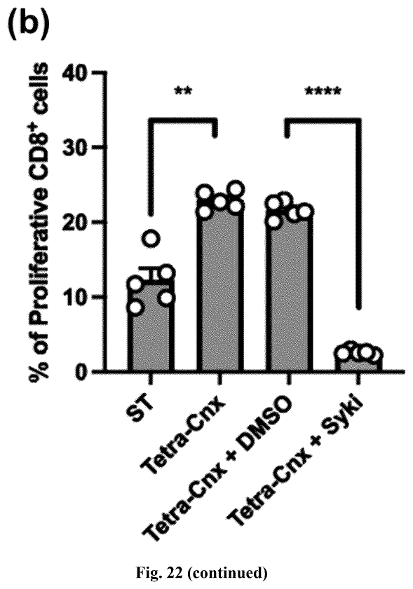

The proliferative capacity of CD8⁺ T cells was evaluated in co-cultures with GM-BMMs pre-treated with either control (ST) or Tetra-CNX, with or without SYK kinase inhibition (FIG. 22). Data are presented as mean±SEM from three biological replicates. Statistical significance was determined using unpaired two-tailed Student's t-test. **P<0.01 and ***P<0.001 were considered statistically significant. Tetra-CNX significantly enhanced CD8⁺ T cell proliferation compared to ST and DMSO-treated groups. However, this enhancement was abolished upon treatment with a SYK inhibitor (Syki), confirming that the observed T cell-stimulatory effect is dependent on SYK signaling. These results provide functional evidence that the immunopotentiating effect of Tetra-CNX on CD8 T cell activation requires downstream TREM1-SYK pathway integrity.

### Example 6. In Vivo Antitumor Activity

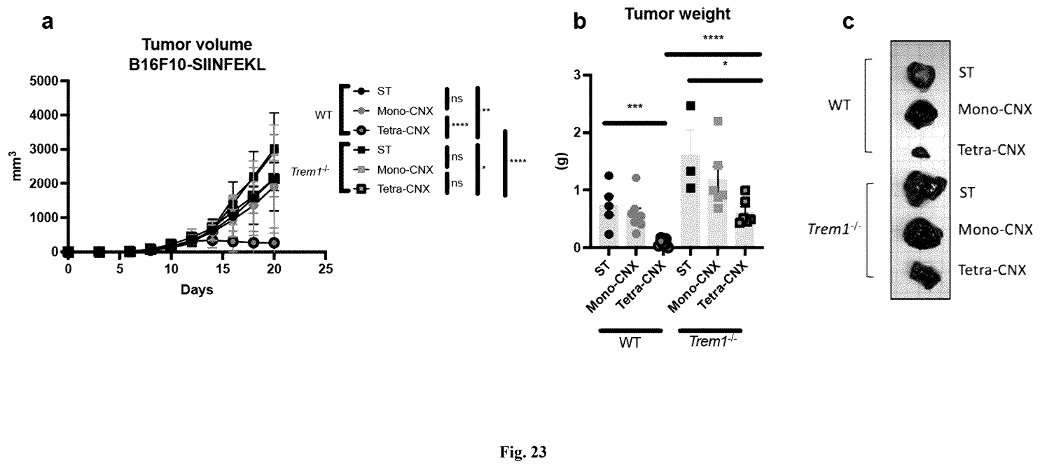

As shown in FIG. 23, the antitumor efficacy of Tetra-CNX was assessed in the B16F10-SIINFEKL melanoma model using both wild-type and Trem1⁻/⁻ mice. In wild-type mice, intratumoral administration of Tetra-CNX significantly reduced tumor volume (FIG. 23a) and tumor weight (FIG. 23b) compared to control and monomeric CNX treatment. Gross excised tumors confirmed this reduction in tumor burden (FIG. 23c). Tumor volumes and weights were compared using unpaired two-tailed Student's t-test. Data represent mean±SEM. In contrast, no therapeutic benefit was observed in Trem1⁻/⁻ mice, indicating that the antitumor effect of Tetra-CNX is dependent on endogenous TREM1 expression. These findings support the functional relevance of the TREM1-ligand axis in mediating myeloid-targeted immunotherapeutic responses in vivo.

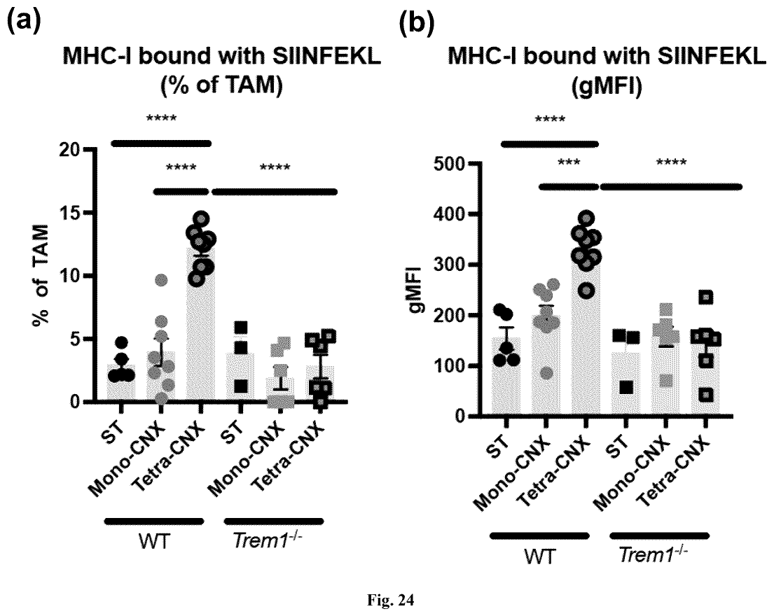

As shown in FIG. 24, Tetra-CNX treatment significantly enhanced the ability of tumor-associated macrophages (TAMs) to present antigenic peptide in the context of MHC class I. Flow cytometric analysis demonstrated a marked increase in the percentage of TAMs positive for SIINFEKL-MHC-I complexes (FIG. 24a) and a corresponding elevation in surface staining intensity (gMFI) (FIG. 24b) following Tetra-CNX administration in wild-type mice. These effects were not observed in Trem1⁻/⁻ mice, supporting a TREM1-dependent mechanism. The data indicate that Tetra-CNX promotes cross-presentation of tumor antigens by TAMs in vivo. Data are shown as mean±SEM. Statistical significance was calculated using unpaired two-tailed Student's t-test.

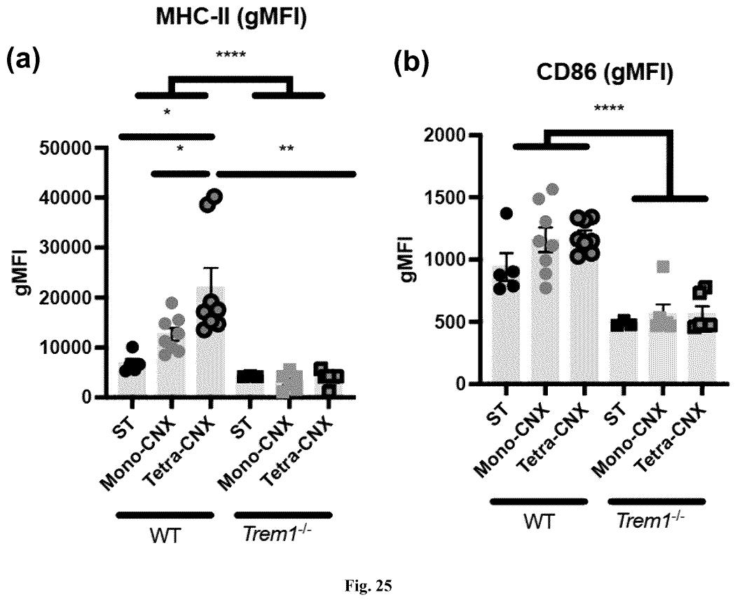

As shown in FIG. 25, Tetra-CNX markedly enhanced the phenotypic activation of tumor-associated macrophages (TAMs) in vivo. Specifically, treatment with Tetra-CNX led to a significant increase in the surface expression of MHC class II molecules (FIG. 25a) and the co-stimulatory marker CD86 (FIG. 25b) in wild-type mice. These effects were absent in Trem1⁻/⁻ animals, indicating that Tetra-CNX-mediated macrophage activation is dependent on TREM1 signaling. The upregulation of MHC-II and CD86 supports the role of Tetra-CNX in promoting a functional antigen-presenting phenotype in TAMs. Data are shown as mean±SEM. Statistical significance was calculated using unpaired two-tailed Student's t-test.

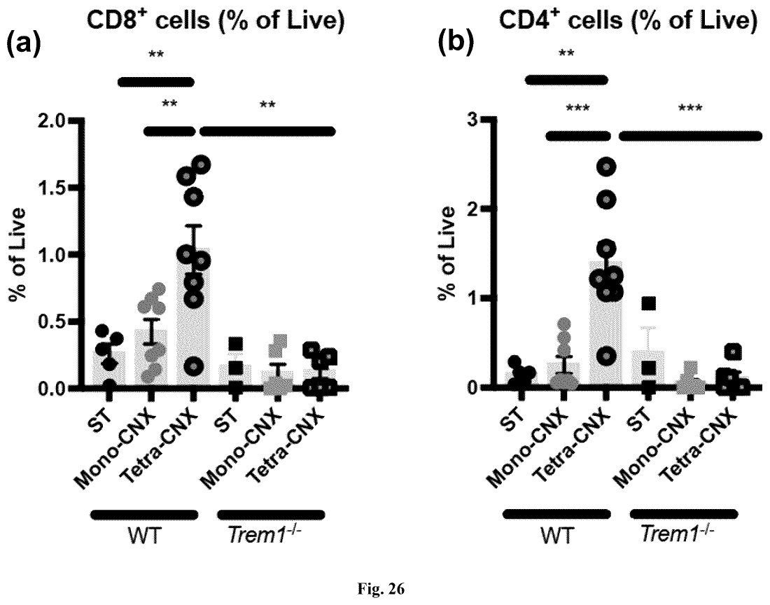

As shown in FIG. 26, administration of Tetra-CNX significantly increased the infiltration of both CD8⁺ and CD4⁺ T cells into the tumor microenvironment of wild-type mice. Flow cytometric analysis revealed a marked elevation in the percentage of CD8⁺ (FIG. 26a) and CD4⁺ (FIG. 26b) T cells among live tumor-infiltrating cells. This effect was abolished in Trem1⁻/⁻ mice, demonstrating the requirement of TREM1 signaling for the Tetra-CNX-mediated enhancement of T cell recruitment. These findings indicate that Tetra-CNX not only reprograms myeloid function but also modulates lymphoid composition within the tumor milieu in a TREM1-dependent manner. Data are shown as mean±SEM. Statistical significance was calculated using unpaired two-tailed Student's t-test.

What is claimed is:

1. A composition comprising a recombinant TREM1 agonist ligand, wherein the recombinant TREM1 agonist ligand comprises a luminal domain in a calnexin assembled capable of binding and activating TREM1 on myeloid cells.

2. The composition of claim 1, wherein the recombinant TREM1 agonist ligand comprises multiple luminal domains in a plurality of calnexins.

3. The composition of claim 2, wherein the recombinant TREM1 agonist ligand further comprises at least four luminal domains in at least four calnexins linked via StrepTagII (SEQ ID NO. 4) and assembled using StrepTactin.

4. The composition of claim 3, wherein the recombinant TREM1 agonist ligand is multivalent.

5. The composition of claim 4, wherein the luminal domain comprises an amino acid sequence set forth in any one of SEQ ID NOs: 1 to 2.

6. A method of enhancing T cells-mediated antitumor immunity in a subject in need thereof, comprising administering to the subject a therapeutically effective amount of the composition of claim 1.

7. The method of claim 6, wherein the T cells are CD8⁺ T cells and CD4⁺ T cells.

8. The method of claim 7, wherein the CD8⁺ T cells are stem-like CD8⁺ T cells or early activated CD8⁺ T cells.

9. The method of claim 7, wherein the CD4⁺ T cells are early activated CD4⁺ T cells or effector CD4⁺ T cells.

10. The method of claim 6, wherein a recombinant TREM1 agonist ligand enhances MHC antigens presentation and antigen processing by macrophages.

11. The method of claim 10, wherein the MHC antigens are MHC class I antigens or MHC class II antigens.

12. The method of claim 10, wherein the macrophages are tumor-associated macrophages.

13. The method of claim 12, wherein the macrophages are derived from myeloid cells.

14. The method of claim 10, wherein the recombinant TREM1 agonist ligand induces phosphorylation of SYK kinase in TREM1-expressing macrophages.

15. The method of claim 10, wherein the recombinant TREM1 agonist ligand enhances phagolysosomal activity in macrophages, as indicated by increased lysosomal acidification, reactive oxygen species production, or antigen degradation.

16. A method for treating cancer in a subject comprising administering to the subject the composition of claim 1, in an amount effective to treat cancer.

17. The method of claim 16, wherein the cancer is diagnosed with a solid tumor.

18. The method of claim 16, further comprising administering an immune checkpoint inhibitor.


SEQUENCE LISTING (4 sequences)

SEQ ID NO: 1 - protein, 461 aa, Homo sapiens
  source 1..461: note = Luminal domain of calnexin from human; identified as a ligand of TREM1
HDGHDDDVIDIEDDLDDVIEEVEDSKPDTTAPPSSPKVTYKAPVPTGEVYFADSFDRGTLSGWILSKAKKDDTDDEIAKYDGKWEVEEMKESKLPGDKGLVLMSRAKHHAISAKLNKPFLFDTKPLIVQYEVNFQNGIECGGAYVKLLSKTPELNLDQFHDKTPYTIMFGPDKCGEDYKLHFIFRHKNPKTGIYEEKHAKRPDADLKTYFTDKKTHLYTLILNPDNSFEILVDQSVVNSGNLLNDMTPPVNPSREIEDPEDRKPEDWDERPKIPDPEAVKPDDWDEDAPAKIPDEEATKPEGWLDDEPEYVPDPDAEKPEDWDEDMDGEWEAPQIANPRCESAPGCGVWQRPVIDNPNYKGKWKPPMIDNPSYQGIWKPRKIPNPDFFEDLEPFRMTPFSAIGLELWSMTSDIFFDNFIICADRRIVDDWANDGWGLKKAADGAAEPGVVGQMIEAAEERP

SEQ ID NO: 2 - protein, 462 aa, Mus musculus
  source 1..462: note = Luminal domain of calnexin from mouse; identified as a ligand of TREM1
HDGHDDDAIDIEDDLDDVIEEVEDSKSKSDASTPPSPKVTYKAPVPTGEVYFADSFDRGSLSGWILSKAKKDDTDDEIAKYDGKWEVDEMKETKLPGDKGLVLMSRAKHHAISAKLNKPFLFDTKPLIVQYEVNFQNGIECGGAYVKLLSKTAELSLDQFHDKTPYTIMFGPDKCGEDYKLHFIFRHKNPKTGVYEEKHAKRPDADLKTYFTDKKTHLYTLILNPDNSFEILVDQSVVNSGNLLNDMTPPVNPSREIEDPEDRKPEDWDERPKIADPDAVKPDDWDEDAPSKIPDEEATKPEGWLDDEPEYIPDPDAEKPEDWDEDMDGEWEAPQIANPKCESAPGCGVWQRPMIDNPNYKGKWKPPMIDNPNYQGIWKPRKIPNPDFFEDLEPFKMTPFSAIGLELWSMTSDIFFDNFIISGDRRVVDDWANDGWGLKKAADGAAEPGVVLQMLEAAEERP

SEQ ID NO: 3 - protein, 39 aa, synthetic construct
  source 1..39: note = Synthetic signal peptide, HA Tag and flexible linker
METDTLLLWVLLLWVPGSTGDYPYDVPDYAGAQPARSTR

SEQ ID NO: 4 - protein, 8 aa, synthetic construct
  source 1..8: note = StrepTagII sequence for purification and Tetramerization
WSHPQFEK